(19) United States
(12) Patent Application Publication
(10) Pub. No.: US 2020/0197065 A1
(43) Pub. Date: Jun. 25, 2020

(54) SURGICAL DEVICE AND SURGICAL IMPACTOR THEREOF

(71) Applicant: Ren-Hong Huang, Miaoli County (TW)

(72) Inventor: Ren-Hong Huang, Miaoli County (TW)

(21) Appl. No.: 16/726,805

(22) Filed: Dec. 24, 2019

Related U.S. Application Data
(60) Provisional application No. 62/784,706, filed on Dec. 25, 2018.

(51) Int. Cl. A61B 17/92

(52) U.S. Cl. CPC A61B 17/92; A61B 2017/924

(57) ABSTRACT

A surgical device includes a surgical instrument and a surgical impactor. The surgical instrument includes a connector whereon a notch is formed. The surgical impactor is configured to be connected to the surgical instrument and includes a shaft, an engaging head, a lever and an operating component. The engaging head is disposed on the shaft for engaging with the connector. The lever is movably disposed on the shaft for engaging with the notch. The operating component is movably disposed on the shaft and includes a wedge-shaped portion movably located between the lever and the shaft. The wedge-shaped portion drives the lever to engage with the notch and eliminate gaps and tolerances between the lever and other structures, including the lever and the notch, during movement of the wedge-shaped portion to a locked position, so as to provide a shock-absorbable and releasable connection between the surgical instrument and the surgical impactor.

### CROSS REFERENCE TO RELATED APPLICATIONS

This application claims the benefit of U.S. Provisional Patent Application No. 62/784,706 filed on Dec. 25, 2018, which is hereby incorporated by reference in its entirety.

### BACKGROUND OF THE INVENTION

### 1. Field of the Invention

The present invention relates to a surgical device, and more particularly, to a surgical device with enhanced operability and reliability and a surgical impactor thereof.

### 2. Description of the Prior Art

An orthopedic surgical instrument, such as a broach, is usually connected with a surgical impaction handle for use. The surgical instrument can be connected to the surgical impaction handle firmly in the beginning. However, after the handle receives repeated forward and backward impacting force during the surgical operation, surgical instrument may be separated from the surgical impaction handle accidentally due to vibration or wear parts of the connection, which not only causes delay in the surgical operation but also increases a risk of surgical site infection.

### SUMMARY OF THE INVENTION

Therefore, an objective of the present invention is to provide a surgical device with enhanced operability and reliability, and a surgical impactor thereof for solving the aforementioned drawbacks.

In order to achieve the aforementioned objective, the present invention discloses a surgical device. The surgical device includes a surgical instrument and a surgical impactor. The surgical instrument includes a connector. A notch is formed on the connector. The surgical impactor is configured to be connected to the surgical instrument. The surgical impactor includes a shaft, an engaging head, a lever and an operating component. The shaft has a proximal end and a distal end. The engaging head is disposed on the distal end of the shaft and for engaging with the connector. The lever is movably disposed on the shaft and for engaging with the notch. The operating component is movably disposed on the proximal end of the shaft. The operating component includes a wedge-shaped portion movably located between the lever and the shaft. The wedge-shaped portion drives the lever to engage with the notch during movement of the wedge-shaped portion toward a locked position. The wedge-shaped portion is engaged by the lever and the shaft to restrain movement of the lever relative to the shaft when the wedge-shaped portion is located at the locked position. Furthermore, the present invention further discloses a related surgical impactor.

For the above, in the present invention, the wedge-shaped portion can eliminate the gaps and tolerances between the lever and other structures, such as the notch and the lever, during movement of the wedge-shaped portion toward the locked position. The lever is engaged with the notch and absorbing shock forces from impaction, which prevents damaged and disengagement of the lever and the notch to ensure a firm connection between the surgical instrument and the surgical impactor. Therefore, in contrast to the prior art, the surgical device of the present invention has enhanced operability and reliability.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram of a surgical device according to a first embodiment of the present invention.

FIG. 2 is an exploded diagram of the surgical device according to the first embodiment of the present invention.

FIG. 3 and FIG. 4 are diagrams of the surgical device at different states according to the first embodiment of the present invention.

FIG. 5 is a schematic diagram of a surgical device according to a second embodiment of the present invention.

FIG. 6 is an exploded diagram of the surgical device according to the second embodiment of the present invention.

FIG. 7 and FIG. 8 are diagrams of the surgical device at different states according to the second embodiment of the present invention.

### DETAILED DESCRIPTION

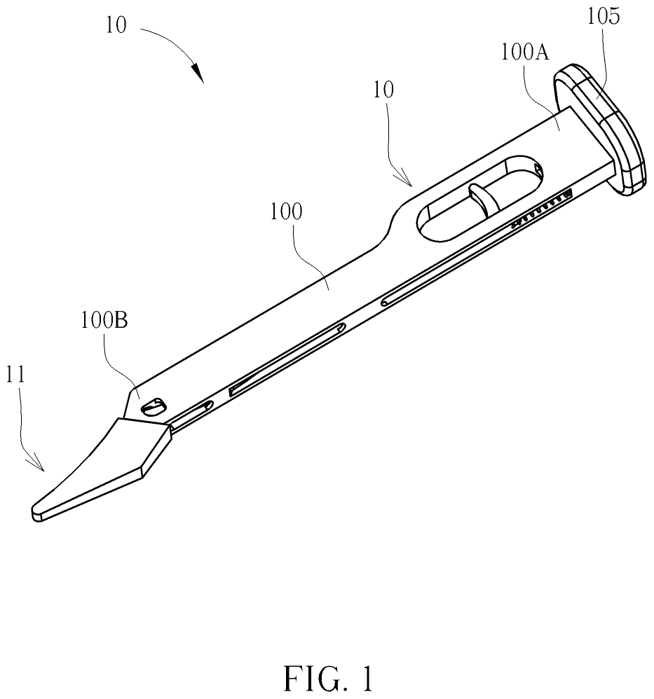

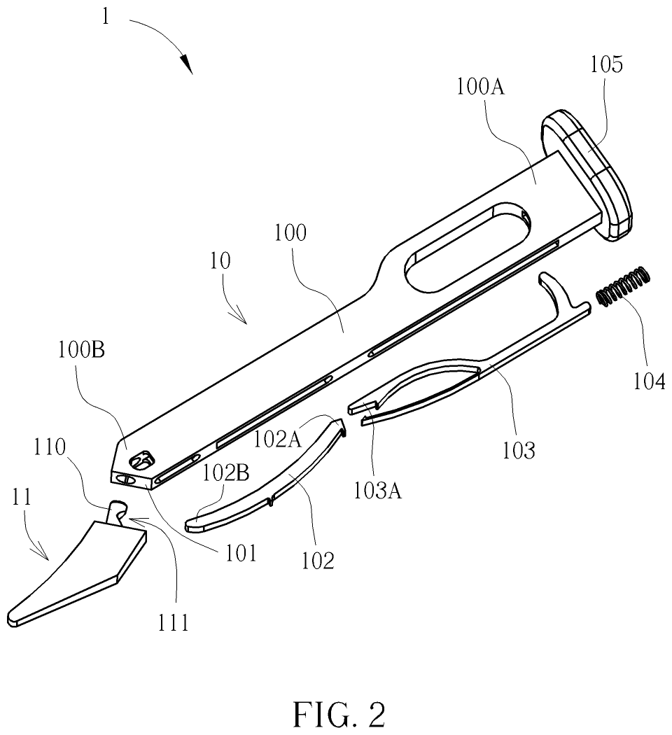

As shown in FIG. 1 to FIG. 4, a surgical device 1 includes a surgical impactor 10 and a surgical instrument 11. In this embodiment, the surgical instrument 11 can be a broach. However, it is not limited thereto, and it could be a different size/shape instrument with a male or female connector. The surgical instrument 11 includes a connector 110. A notch 111 is formed on connector 110. The surgical impactor 10 is configured to be connected to surgical instrument 11. The surgical impactor 10 includes a shaft 100, an engaging head 101, a lever 102, an operating component 103, a resilient component 104 and an impaction plate 105. The shaft 100 has a proximal end 100A and a distal end 100B. The impaction plate 105 is disposed on proximal end 100A of shaft 100 for receiving an external impacting force. The impaction plate 105 allows a user to apply a force for surgical device 1. The engaging head 101 is disposed on distal end 100B of shaft 100 and for engaging with connector 110. The lever 102 is movably disposed on distal end 100B of shaft 100 and for engaging with notch 111. The operating component 103 is movably disposed on proximal end 100A of shaft 100. The operating component 103 can be operated to allow or restrain disengagement of lever 102 and notch 111. The resilient component 104 is disposed between operating component 103 and shaft 100 to bias operating component 103 to restrain the disengagement of lever 102 and notch 111.

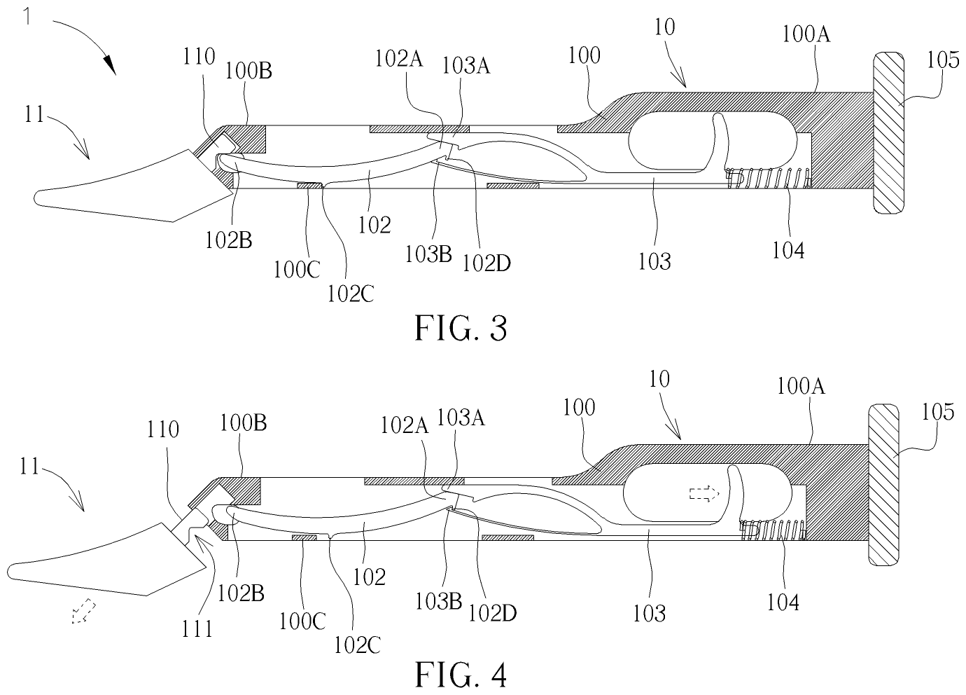

Specifically, the shaft 100 can be a straight stem structure, and operating component 103 and lever 102 are slidably disposed on the shaft 100. The operating component 103 includes a wedge-shaped portion 103A movably located between lever 102 and shaft 100 for engaging with lever 102 and shaft 100. The lever 102 includes a first engaging portion 102A and a second engaging portion 102B. The first engaging portion 102A is for abutting against wedge-shaped portion 103A. The second engaging portion 102B is opposite to first engaging portion 102A and for engaging with notch 111. The wedge-shaped portion 103A can be moved between a locked position as shown in FIG. 3 and a released position as shown in FIG. 4 when operating component 103 is operated. During movement of wedge-shaped portion 103A toward the locked position, the wedge-shaped portion 103A abuts first engaging portion 102A to drive lever 102 to move toward distal end 100B of shaft 100 to engage second engaging portion 102B with notch 111. Furthermore, the wedge-shaped portion 103A is engaged by lever 102 and shaft 100 to restrain movement of lever 102 relative to shaft 100 when wedge-shaped portion 103A is located at the locked position.

It should be noticed that resilient component 104 is resiliently deformed to generate a continuous force for driving operating component 103 to ensure engagement of wedge-shaped portion 103A, the first engaging portion 102A of lever 102 and shaft 100, so as to ensure engagement of second engaging portion 102B of lever 102 and notch 111 when wedge-shaped portion 103A is located at the locked position. Therefore, it ensures a firm connection between surgical instrument 11 and surgical impactor 10 when impaction plate 105 receives an external impacting force during a surgical operation.

Preferably, in this embodiment, the lever 102 can further include a stopping portion 102C disposed between first engaging portion 102A and second engaging portion 102B, and stopping portion 102C abuts against a protrusion 100C of shaft 100 when wedge-shaped portion 103A is located at the locked position, which achieves a purpose of positioning wedge-shaped portion 103A at the locked position.

On the other hand, when operating component 103 is operated to drive wedge-shaped portion 103A to move from the locked position as shown in FIG. 3 to the released position as shown in FIG. 4, the engagement of wedge-shaped portion 103A, first engaging portion 102A of lever 102 and shaft 100 is released, so that lever 102 is allowed to move toward proximal end 100A of shaft 100 for disengaging second engaging portion 102B from notch 111. When lever 102 moves to disengage second engaging portion 102B from notch 111, the surgical instrument 11 can be detached from surgical impactor 10.

Preferably, in this embodiment, in order to facilitate detachment operation of surgical instrument 11, operating component 103 can further include a driving portion 103B. The lever 102 further includes a driven portion 102D adjacent to first engaging portion 102A. The driving portion 103B abuts against driven portion 102D to drive lever 102 to move toward proximal end 100A of shaft 100 to disengage second engaging portion 102B from notch 111 after wedge-shaped portion 103A is moved to leave from the locked position. Furthermore, in this embodiment, the driving portion 103B can be an engaging hook with a resilient arm, and driven portion 102D can be an engaging protrusion, so that driving portion 103B can be resiliently deformed by driven portion 102D of lever 102 for preventing interference between driving portion 103B and lever 102 when wedge-shaped portion 103A moves to the locked position. However, it is not limited thereto.

Besides, preferably, in this embodiment, the lever 102 can be an elongated rod structure, so that lever 102 can be forced to be resiliently bent for preventing structural damage caused by the impacting force transmitted from impaction plate 105. For example, when it is desired to remove surgical instrument 11 stuck in a patient's body by a hammer, the lever 102 can be forced by connector 110 to be resiliently bent for preventing crack or damage of connector 110.

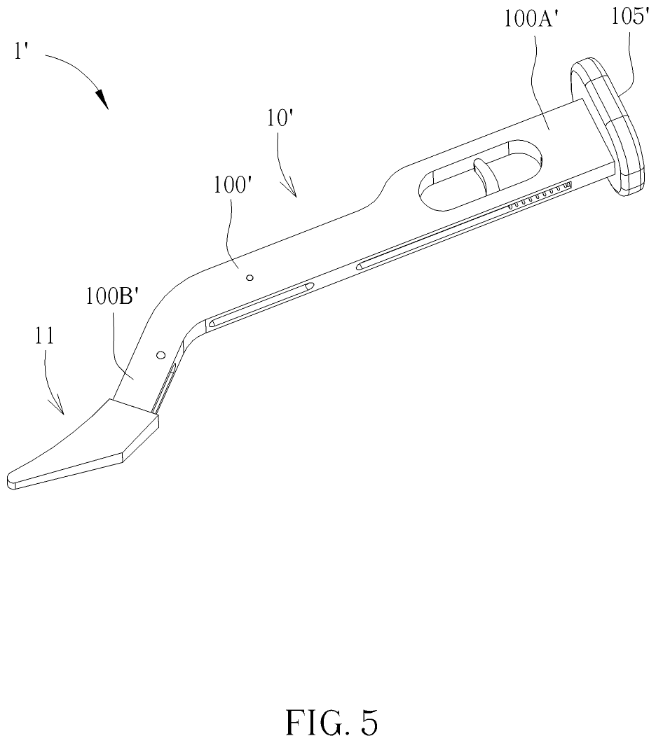

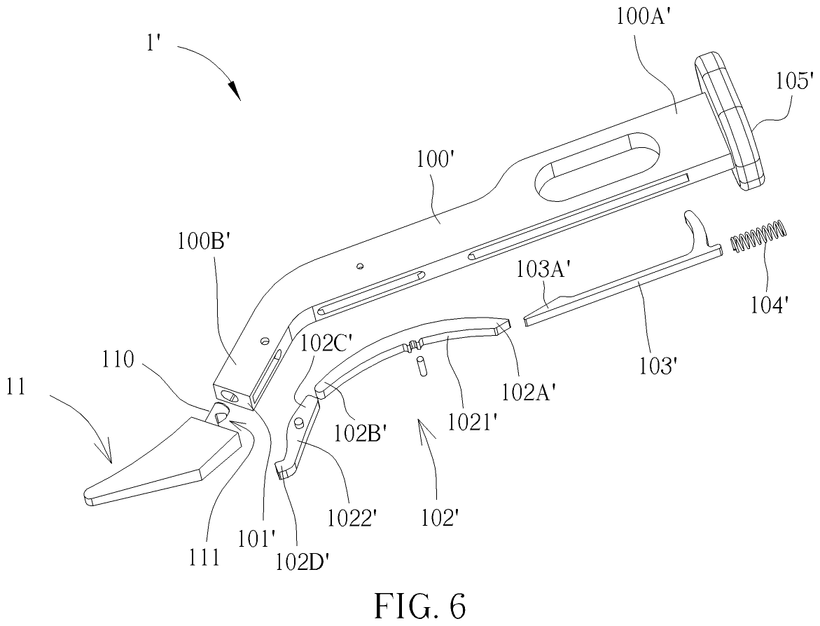

As shown in FIG. 5 to FIG. 8, different from surgical device 10′ of the first embodiment, a surgical device 1′ includes a surgical impactor 10′ and surgical instrument 11. The surgical impactor 10′ includes a shaft 100′, an engaging head 101′, a lever 102′, an operating component 103′, a resilient component 104′ and an impaction plate 105′.

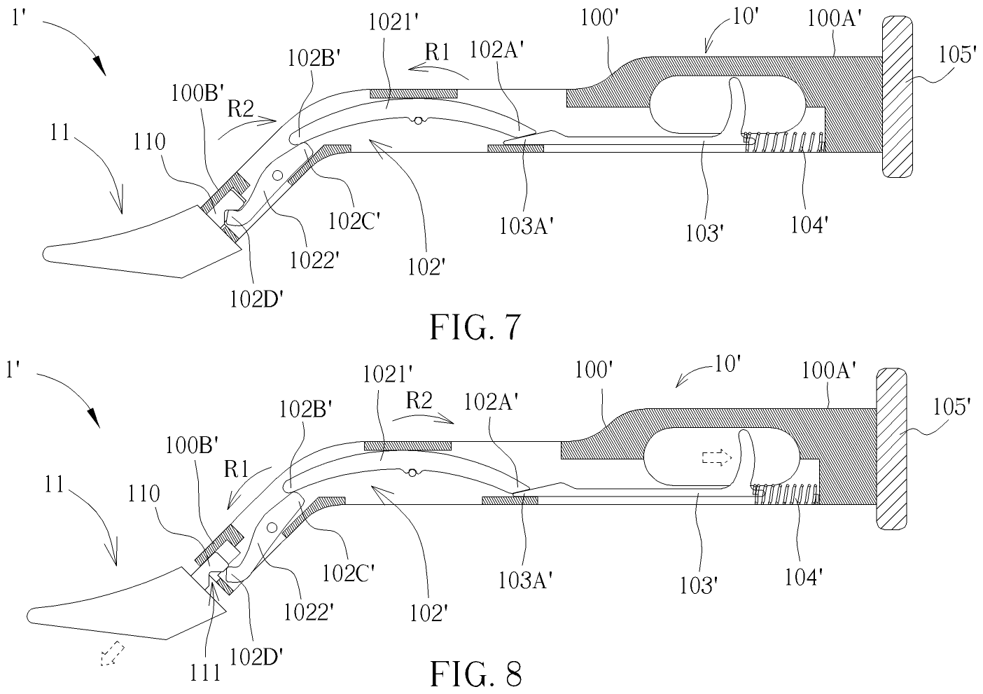

The shaft 100′ can be a curved structure and has a proximal end 100A′ and a distal end 100B′. The impaction plate 105′ is disposed on proximal end 100A′ of shaft 100′ for receiving an external impacting force. The impaction plate′ 105 allows a user to apply a force for surgical device 1′. The engaging head 101′ is disposed on distal end 100B′ of shaft 100′ and for engaging with connector 110. The operating component 103′ is slidably disposed on shaft 100′ and includes a wedge-shaped portion 103A′ movable between a locked position as shown in FIG. 7 and a released position as shown in FIG. 8. The resilient component 104′ is disposed between operating component 103′ and shaft 100′ to bias operating component 103′ to restrain the disengagement of lever 102′ and notch 111 formed on connector 110. The lever 102′ includes a lever element 1021′ and a hook element 1022′. The lever element 1021′ and hook element 1022′ are pivotally disposed on shaft 100′. The lever element 1021′ includes a first engaging part 102A′ and a second engaging part 102B′ opposite to first engaging part 102A′. The hook element 1022′ includes a third engaging part 102C′ and a fourth engaging part 102D′ opposite to third engaging part 102C′. The first engaging part 102A′ is configured to abut against wedge-shaped portion 103A′. The second engaging part 102B′ is configured to abut against third engaging part 102C′, and fourth engaging part 102D′ is configured to engage with notch 111. When lever element 1021′ pivots relative to shaft 100′ in a first pivoting direction R1, the hook element 1022′ is driven by lever element 1021′ to pivot relative to shaft 100′ in a second pivoting direction R2 opposite to first pivoting direction R1.

When operating component 103′ is operated to move wedge-shaped portion 103A′ toward the locked position as shown in FIG. 7, the lever element 1021′ is driven by abutment of first engaging part 102A′ and wedge-shaped portion 103A′ to pivot in first pivoting direction R1 for driving hook element 1022′ to pivot in second pivoting direction R2 by abutment of second engaging part 102B′ and third engaging part 102C′, so as to engage fourth engaging part 102D′ with notch 111. When wedge-shaped portion 103A′ is located at the locked position, the wedge-shaped portion 103A′ is engaged by lever element 1021′ and shaft 100′ to restrain pivotal movement of lever element 1021′ relative to shaft 100′.

It should be noticed that resilient component 104′ is resiliently deformed to generate a continuous force for driving operating component 103′ to ensure engagement of wedge-shaped portion 103A′, the first engaging part 102A′ of lever element 1021′ and shaft 100′, so as to ensure engagement of fourth engaging part 102D′ and notch 111 when wedge-shaped portion 103A′ is located at the locked position. Therefore, it ensures a firm connection between surgical instrument 11 and surgical impactor 10′ when impaction plate 105′ receives an external impacting force during a surgical operation.

On the other hand, when operating component 103′ is operated to drive wedge-shaped portion 103A′ to move from the locked position as shown in FIG. 7 to the released position as shown in FIG. 8, the engagement of wedge-shaped portion 103A′, the first engaging part 102A′ of lever element 1021′ and shaft 100′ is released, so that lever element 1021′ is allowed to pivot in second pivoting direction R2 for allowing disengagement of fourth engaging part 102D′ and notch 111 by pivoting hook element 1022′ in first pivoting direction R1. For example, the hook element 1022′ can be driven to pivot in first pivoting direction R1 to disengage fourth engaging part 102D′ from notch 111 manually or by a resilient component, such as a torsional spring. When hook element 1022′ pivots to disengage fourth engaging part 102D′ from notch 111, the surgical instrument 11 can be detached from surgical impactor 10′.

Besides, in this embodiment, in order to prevent structural damage caused by the impacting force transmitted from impaction plate 105, the lever element 1021′ can be preferably an elongated rod structure, so that lever element 1021′ can be forced to be resiliently bent.

For the above, in the present invention, the wedge-shaped portion can eliminate the gaps and tolerances between the lever and other structures, such as the lever and the notch, during movement of the wedge-shaped portion toward the locked position. The lever is engaged with the notch and absorbing shock forces from impaction, which prevents damaged and disengagement of the lever and the notch to ensure a firm connection between the surgical instrument and the surgical impactor. Therefore, in contrast to the prior art, the surgical device of the present invention has enhanced operability and reliability.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A surgical device comprising:
a surgical instrument comprising a connector, a notch being formed on the connector; and
a surgical impactor configured to be connected to the surgical instrument, the surgical impactor comprising:
a shaft having a proximal end and a distal end;
an engaging head disposed on the distal end of the shaft and for engaging with the connector;
a lever movably disposed on the shaft and for engaging with the notch; and
an operating component movably disposed on the proximal end of the shaft, the operating component comprising a wedge-shaped portion movably located between the lever and the shaft, the wedge-shaped portion driving the lever to engage with the notch during movement of the wedge-shaped portion toward a locked position, the wedge-shaped portion being engaged by the lever and the shaft to restrain movement of the lever relative to the shaft when the wedge-shaped portion is located at the locked position.

2. The surgical device of claim 1, wherein the lever comprises:
a first engaging portion for abutting against the wedge-shaped portion; and
a second engaging portion opposite to the first engaging portion and for engaging with the notch.

3. The surgical device of claim 2, wherein the operating component further comprises a driving portion, the lever further comprises a driven portion adjacent to the first engaging portion, and the driving portion abuts against the driven portion to disengage the second engaging portion from the notch after the wedge-shaped portion is moved to leave from the locked position.

4. The surgical device of claim 2, wherein the lever further comprises a stopping portion disposed between the first engaging portion and the second engaging portion, and the stopping portion abuts against a protrusion of the shaft when the wedge-shaped portion is located at the locked position.

5. The surgical device of claim 2, wherein the operating component and the lever are slidably disposed on the shaft.

6. The surgical device of claim 1, wherein the lever comprises a lever element and a hook element, the lever element comprises a first engaging part and a second engaging part opposite to the first engaging part, the hook element comprises a third engaging part and a fourth engaging part opposite to the third engaging part, the first engaging part is configured to abut against the wedge-shaped portion, the second engaging part is configured to abut against the third engaging part, and the fourth engaging part is configured to engage with the notch.

7. The surgical device of claim 6, wherein the operating component is slidably disposed on the shaft, the lever element and the hook element are pivotally disposed on the shaft, and when the lever element pivots relative to the shaft in a first pivoting direction, the hook element is driven by the lever element to pivot relative to the shaft in a second pivoting direction opposite to the first pivoting direction.

8. The surgical device of claim 1, wherein the surgical impactor further comprises a resilient component disposed between the operating component and the shaft to bias the wedge-shaped portion to move toward the locked position.

9. The surgical device of claim 1, wherein the surgical impactor further comprises an impaction plate disposed on the proximal end of the shaft.

10. A surgical impactor configured to be connected to a surgical instrument, the surgical impactor comprising:
a shaft having a proximal end and a distal end;
an engaging head disposed on the distal end of the shaft and for engaging with a connector of the surgical instrument;
a lever movably disposed on the shaft and for engaging with a notch on the connector of the surgical instrument; and
an operating component movably disposed on the proximal end of the shaft, the operating component comprising a wedge-shaped portion movably located between the lever and the shaft, the wedge-shaped portion driving the lever to engage with the notch during movement of the wedge-shaped portion toward a locked position, the wedge-shaped portion being engaged by the lever and the shaft to restrain movement of the lever relative to the shaft when the wedge-shaped portion is located at the locked position.

11. The surgical impactor of claim 10, wherein the lever comprises:
a first engaging portion for abutting against the wedge-shaped portion; and
a second engaging portion opposite to the first engaging portion and for engaging with the notch.

12. The surgical impactor of claim 11, wherein the operating component further comprises a driving portion, the lever further comprises a driven portion adjacent to the first engaging portion, and the driving portion abuts against the driven portion to disengage the second engaging portion from the notch after the wedge-shaped portion is moved to leave from the locked position.

13. The surgical impactor of claim 11, wherein the lever further comprises a stopping portion disposed between the first engaging portion and the second engaging portion, and the stopping portion abuts against a protrusion of the shaft when the wedge-shaped portion is located at the locked position.

14. The surgical impactor of claim 11, wherein the operating component and the lever are slidably disposed on the shaft.

15. The surgical impactor of claim 10, wherein the lever comprises a lever element and a hook element, the lever element comprises a first engaging part and a second engaging part opposite to the first engaging part, the hook element comprises a third engaging part and a fourth engaging part opposite to the third engaging part, the first engaging part is configured to abut against the wedge-shaped portion, the second engaging part is configured to abut against the third engaging part, and the fourth engaging part is configured to engage with the notch.

16. The surgical impactor of claim 15, wherein the operating component is slidably disposed on the shaft, the lever element and the hook element are pivotally disposed on the shaft, and when the lever element pivots relative to the shaft in a first pivoting direction, the hook element is driven by the lever element to pivot relative to the shaft in a second pivoting direction opposite to the first pivoting direction.

17. The surgical impactor of claim 10, further comprising a resilient component disposed between the operating component and the shaft to bias the wedge-shaped portion to move toward the locked position.

18. The surgical impactor of claim 10, further comprising an impaction plate disposed on the proximal end of the shaft.
